(19) United States
(12) Patent Application Publication
(10) Pub. No.: US 2011/0059179 A1
(43) Pub. Date: Mar. 10, 2011

(54) CONTROLLED DELIVERY SYSTEM THAT PROVIDES MALODOR MANAGEMENT OVER A PROLONGED PERIOD OF TIME

(76) Inventors: Adi Shefer, East Brunswick, NJ (US); Samuel Shefer, East Brunswick, NJ (US)

(21) Appl. No.: 12/555,088

(22) Filed: Sep. 8, 2009

(51) Int. Cl. A61K 9/50; A61P 43/00; A61L 9/012; A61Q 13/00

(52) U.S. Cl. 424/490; 510/119; 510/130; 424/76.2

(57) ABSTRACT

The present invention relates to an improved controlled delivery system that can be incorporated into personal and household care products, such as deodorants, body wash, hair products, air refresher, fabric refresher, and other personal and household products, for the counteraction and management of malodor. The controlled delivery system of the present invention controls malodor by a dual action; it absorbs and neutralizes the malodor and it releases an odorous or non-odorous substance to counteract malodor in the environment over an extended period of time. The simultaneous dual action of the controlled delivery system of the present invention eliminates the malodor and can provide a long lasting pleasant odor over an extended period of time.

### BACKGROUND OF THE INVENTION

1. Field of the Invention

The present invention relates to an improved controlled delivery system that can be incorporated into personal and household care products, such as deodorants, body wash, hair products, air refresher, fabric refresher, and other personal and household products, for the counteraction and management of malodor. The controlled delivery system of the present invention controls malodor by a dual action; it absorbs and neutralizes the malodor and it releases an odorous or non-odorous substance to counteract malodor in the environment over an extended period of time. The simultaneous action of the controlled delivery system of the present invention eliminates the malodor and can provide a long lasting pleasant odor over an extended period of time.

2. Description of the Related Art

A major effort in the art of perfumery has been directed to providing means of treating odors that are offensive to the human sense of smell, i.e., malodor. The odors encompass a variety of odors such as bathroom-odor, kitchen-odor, body-odor, cigar smoke-odor, and the like. Conventional products, including fresheners and deodorizers, suggest solutions to overcome and combat these malodors. In general, the conventional products provide a masking effect by one of two mechanisms. Masking the malodor with a fragrance that either suppresses the offensive odor by providing a more pleasing aroma in larger quantities or creating a fragrance that blends with the offensive odor to provide a different, more desirable aroma. Unfortunately, in both instances a large amount of fragrance must be utilized which in itself often proves to be offensive. Furthermore, the offensive odor is usually still detectable.

Attempts have also been made to control malodors through the use of odor absorbents. Zeolites such as those marketed under the name ABSCENTS™ by Union Carbide Corporation and UOP LLC, are known odor absorbers. However these solid odor absorbers, as well as activated charcoal odor absorbers, lose functionality when wet. Therefore, these malodor absorbers are not effective applications where the product containing them is likely to be wetted by body fluids as they lose their desired odor absorbent characteristics. Furthermore, zeolites can cause a “harsh” feel if too much is deposited onto the skin.

Cyclodextrines have also been widely used for malodor management and fragrance delivery. Several patents disclose the use of controlled release systems based on cyclodextrin complexes for malodor applications. U.S. Pat. No. 7,109,324 discloses a process for chemically bonding an odor-encapsulating agent to textiles includes reacting a cyclodextrin with a cross-linking agent capable of forming ether bonds with the cyclodextrin and with the textile material, and curing the textile material treated with a mixture of the cyclodextrin and cross-linking agent. Preferably, the cross-linking agent is imidazolidone, which forms an ether bond with a hydroxyl group on the cyclodextrin and with a hydroxyl group. In textile materials containing cellulose, imidazolidone forms an ether bond with a hydroxyl group on the cellulose. An active agent can be complexed with the cyclodextrin for release. Textiles formed by such a process can comprise an article of clothing adapted to trap odors emanating from a wearer.

U.S. Pat. No. 6,689,378 discloses methods of immobilizing uncomplexed and complexed cyclodextrins to cellulose fibers and compositions including cyclodextrin immobilized to cellulose fibers are provided. The cyclodextrins are immobilized using a cross-linking means that covalently bonds the cyclodextrin to the substrate, without the cyclodextrin being derivatized. The compositions can be used in all types of cellulose fiber containing articles, such as tissues and personal care articles, for odor absorbency or for release of complexed molecules. U.S. Pat. Nos. 5,660,845 and 5,783,552 also disclose the use of cyclodextrin for odor absorption.

Cyclodextrins are cyclic carbohydrates that can be produced enzymatically from starch, are generally safe for humans, and are non-polluting. In general, cyclodextrins include glucose units arranged in a donut-shaped ring. The specific coupling and conformation of the glucose units give the cyclodextrins a rigid, conical molecular structure with a hollow interior of a specific volume. Cyclodextrins thus offer a toroidal geometry with a hydrophilic exterior, which allows the cyclodextrin to dissolve in water. The “lining” of the internal cavity is formed by hydrogen atoms and glycosidic bridging oxygen atoms. This interior surface, therefore, is fairly hydrophobic. Cyclodextrins have useful chemical properties often tied to the inclusion of chemicals in the toroidal cavity. This cavity can be filled with all or a portion of an organic molecule with suitable size to form an “inclusion complex.” The association of the molecule with the cyclodextrin isolates the molecule from the aqueous solvent and may increase the molecule's stability and water solubility, since the solubility of the cyclodextrin is often higher than the solubility of the molecule. The unique shape and physical-chemical properties of the cavity also enable cyclodextrin molecules to absorb (form inclusion complexes with) organic molecules or parts of organic molecules that can fit into the cavity. Many odorous molecules can fit into the cavity including many malodorous molecules and perfume molecules. Therefore, cyclodextrins, and especially mixtures of cyclodextrins with different size cavities, have been used to control odors caused by a broad spectrum of organic odoriferous materials, which may or may not contain reactive functional groups. The major disadvantage of cyclodextrines is their low payload of aroma chemicals which can be lower than 20% payload by weight thus making them expensive for applications in consumer care products.

U.S. Pat. No. 6,375,966 discloses a polyurethane/urea matrix for the delivery of active agents. The polyurethane/urea matrix is prepared by a process of reacting a urethane prepolymer with an aromatic diamine chain extender in the presence of an active agent which is a fragrance agent or an insect repellant agent. This invention also relates to a process for making the polyurethane/urea matrices as well as the products in which the polyurethane/urea matrices may be incorporated.

U.S. Patent Application Publication No. 2009/0067760 discloses bags or liners having incorporated therein an effective amount of odor management agents including a counteractant agent, a neutralizing agent and optionally, a masking agent, whereby the odor management agent imparts no perceptible scent to the bags or liners, such that the bags or liners are substantially free of fragrance while the odor management agent reduces malodor emanating from products disposed within the bag. The odor management agent is disposed within the bag or dispersed substantially uniformly throughout the web. It is described that controlled delivery can be achieved, for example, by incorporating the odor management agent in the form of a masterbatch, such as during an extrusion process for an extruded web which forms the panels of the bags or liners.

U.S. Pat. Nos. 5,066,419, and 5,154,842 disclose coated perfume particles. The perfume particles comprise perfume dispersed within certain water-insoluble non-polymeric carrier materials and encapsulated in a protective shell by coating with a friable coating material. The coated particles allow for preservation and protection of perfumes, which are susceptible to degradation or loss in storage and in cleaning compositions. In use, the surface coating fractures and the underlying carrier/perfume particles efficiently deliver a large variety of perfume types to fabrics or other surfaces.

U.S. Pat. No. 7,338,928 discloses a controlled release system that can be incorporated in cosmetic, personal care, and household products to effectively encapsulate wide range of active ingredients and sensory markers and release them in response to moisture or over an extended period of time. The controlled release system consists of oil absorbing polymer nanospheres coated with water sensitive surface active polymers. The triggered release system is a dispersion formed of oil absorbing polymer nanospheres comprising sensory markers, and other various active ingredients, surrounded, encapsulated and coated by a surface active moisture sensitive polymer. The controlled release system is characterized by: (i) protection of the active ingredients, as well as the volatile constituents of fragrances, during storage, until needed; (ii) moisture triggered controlled release of the fragrances, and other active ingredients; and (iii) prolonged release of fragrances, and other active ingredients over an extended period of time. The oil-absorbing polymer nanospheres have an average particle diameter that ranges from 20 nm to 1000 nm.

U.S. Pat. Nos. 7,208,460 and 6,825,161 disclose a controlled delivery system that can be incorporated in soap bars to enhance deposition of active ingredients and sensory markers onto skin. The carrier system also provides controlled release or prolonged release of these actives from the skin over an extended period of time. The controlled delivery system comprises substantially free-flowing, powder formed of solid hydrophobic, positively charged, nanospheres of encapsulated active ingredients, that are encapsulated in moisture sensitive microspheres. The high cationic charge density of the nanosphere improves deposition of active ingredients onto skin. The high cationic charge density on the nanosphere surface is created by incorporating a cationic conditioning agent into the solid hydrophobic matrix of the nanospheres, by incorporating a cationic charge “booster” in the moisture sensitive microsphere matrix, or by using a cationic conditioning agent in the nanosphere matrix in conjunction with a cationic charge “booster” in the microsphere matrix. The controlled release system can be included in soap products.

The prior art of which applicant is aware does not set forth a controlled delivery system that can provide malodor management by a dual action; absorbing and neutralizing the malodor and releasing an odorous or non-odorous substance to counteract malodor in the environment over an extended period of time, in a cost effective manner. The present invention addresses the foregoing need to eliminate or reduce offensive malodors from the body, hair, fabric, and other objects in the environment so that the air in the environment remains aesthetically pleasing by employing an advanced controlled delivery system that absorbs malodor and releases malodor counteracting substances.

### SUMMARY OF THE INVENTION

The present invention relates to an improved controlled delivery system for personal and household care products, such as deodorants, body wash, and hair products, air refresher, fabric refresher, and other personal and household products, for the counteraction and management of malodor. The controlled delivery system of the present invention controls malodor by a dual action; it absorbs and neutralizes the malodor and it releases in a controlled manner an odorous or non-odorous substance to counteract malodor in the environment over an extended period of time. The simultaneous action of the delivery system of the present invention eliminates the malodor and can provide a long lasting pleasant odor over an extended period of time.

The controlled delivery system of the present invention consists of spheres composed of a free volume hydrophobic matrix core and a hydrophilic film forming surface. Malodor counteracting fragrances and non-odorous malodor counteracting substances can be encapsulated in the sphere free volume hydrophobic matrix core, at very high payload, up to 60% of the system weight. The film forming hydrophilic surface controls the release rate of the encapsulated substance into the environment over an extended period of time. Upon application of products comprising the controlled delivery system of the present invention, the spheres release the fragrance or the non-odorous malodor counteracting substance into the environment and the free volume created within the spheres upon the release of the encapsulated substance is replaced with the malodor absorbed from the environment. The controlled delivery system of the present invention is very versatile and can be incorporated in water, alcohol, and hydro alcoholic products.

The incorporation of a controlled delivery system comprising fragrances and other non-odorous malodor counteracting substances encapsulated within the sphere hydrophobic free volume matrix core was found to significantly reduce malodor and eliminate it over an extended period of time. In an alternate embodiment, a controlled delivery system was also found to provide in addition to reducing malodor a long lasting pleasing odor.

The invention also provides personal and household care products such as deodorants, body wash, and hair products, fabric refresher, fabric softener, air fresher, and other personal and household products, comprising the controlled delivery system of the present invention. Objects and surfaces releasing malodor treated with products comprising the controlled delivery system of the present invention were observed to exhibit very low malodor and if a fragrance was encapsulated in the controlled delivery system, the treated objects exhibited high level of fragrance (high odor intensity) over an extended period of time, such as a few days to few weeks.

The present invention addresses the foregoing need to eliminate or reduce offensive malodors from the body, hair, fabric, and other objects in the environment so that the air in the environment remains aesthetically pleasing by employing an advanced controlled delivery system that absorbs malodor and releases malodor counteracting substances.

It is believed that the free volume hydrophobic matrix core of the spheres becomes associated, in use of the composition, with malodor ingredients in the environment of treated surfaces or objects and assists in absorbing them into the structure of the sphere, thus eliminating them from the treated surfaces, objects, or environment. The film forming hydrophilic surface of the spheres sustains the diffusion rate of the odorous or the non-odorous substances and enables them to be released from the treated surfaces and objects over an extended period of time.

The controlled release system of the present invention can comprises from about 1% to about 50% by weight free volume hydrophobic matrix core, from about 1% to about 50% by weight film forming hydrophilic surface, from about 1% to about 80% by weight odorous or non-odorous malodor counteracting substances, and from about 1% to about 90% by weight water. The spheres have an average particle size in the range from about 0.001 microns to about 5 microns. The spheres can be incorporated into any personal and household products, preferably in deodorants, body wash, hair products, fabric fresher, and air fresher compositions.

The invention will be more fully described by reference to the following drawings.

### BRIEF DESCRIPTION OF THE DRAWINGS

FIG. 1 is a graph of malodor intensity over time for socks treated with the controlled delivery system of the present invention with cotton fresh fragrance and untreated socks.

FIG. 2 is a graph of malodor intensity over time for socks treated with the controlled delivery system with linen fragrance and untreated socks.

FIG. 3 is a GC-MS chromatogram of onion malodor with peaks at 7.8 minutes and 10.7 minutes.

FIG. 4 is a GC-MS chromatogram of onion malodor before and following treatment with the controlled delivery system.

FIG. 5 is a GC-MS chromatogram of onion malodor before and following treatment with the controlled delivery system.

FIG. 6 is a graph of malodor absorption kinetics.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

The present invention provides a method of controlling offensive malodor in the environment. The controlled delivery system of the present invention is a water dispersion comprising spheres composed of free volume hydrophobic matrix core and having film forming hydrophilic surfaces. The spheres can comprise odorous or non-odorous malodor counteracting substances encapsulated in the sphere hydrophobic matrix core. The term “spheres” is intended to describe spherical particulates. It will be appreciated that other spherical shapes can be used in accordance with the teachings of the present invention.

Spheres of the present invention containing malodor counteracting odorous or non-odorous malodor counteracting substances have an average diameter in the range of from about 0.001 micron to about 5 microns. Preferably, the sphere size is in the range from about 0.05 microns to about 2 microns. This linear dimension for any individual sphere represents the length of the longest straight line joining two points on the surface of the sphere.

The controlled delivery system of the present invention comprises spheres composed of a free volume hydrophobic matrix core and a hydrophilic film forming surface. Odorous and non-odorous malodor counteracting substances are encapsulated in the sphere free volume hydrophobic matrix core. The odorous and non-odorous malodor counteracting substances can be present at very high payload, up to 60% of the weight of the system. For example, the odorous and non-odorous malodor counteracting substances can be present in the range of about 30%-60% or 50%-60% of weight of the system. The film forming hydrophilic surface controls the release rate of the encapsulated substances into the environment over an extended period of time. Upon application of products comprising the controlled delivery system of the present invention, the spheres release the odorous or the non-odorous malodor counteracting substances into the environment and the free volume created within the spheres upon the release of the encapsulated substances is replaced with the malodor absorbed from the environment. The controlled delivery system of the present invention is very versatile and can be incorporated in water, alcohol, and hydro alcoholic products.

The incorporation of the controlled delivery system comprising odorous and/or non-odorous malodor counteracting substances encapsulated within the sphere hydrophobic free volume matrix core was found to significantly reduce malodor and eliminate it over an extended period of time. In an alternate embodiment, a controlled delivery system of the present invention was found to provide in addition to reducing malodor a long lasting pleasing odor.

The invention also provides personal and household products such as deodorants, body wash, and hair products such as shampoos, rinses and conditioners, fabric refresher, fabric softener, air fresher, air refresher and other personal and household products, comprising the controlled delivery system of the present invention. Objects and surfaces releasing malodor treated with the controlled delivery system of the present invention were observed to exhibit very low malodor and if a fragrance was encapsulated in the controlled delivery system, the treated objects exhibited high level of fragrance (high odor intensity) over an extended period of time, such as a few days to few weeks.

It is believed that the free volume hydrophobic matrix of spheres core becomes associated, in use of the composition, with the malodor ingredients in the environment of the treated surfaces or objects and assists in absorbing them into their structure, thus eliminating them from the treated surfaces, objects, or environment. The film forming hydrophilic surface of the spheres sustains the diffusion rate of the odorous or the non-odorous substances and enables them to be released from the treated surfaces and objects over an extended period of time.

### Matrix Materials for Forming Spheres

Materials for the free volume hydrophobic matrix core of the spheres of the present invention comprises of water-insoluble particulate heteropolymers made by sequential emulsion polymerization including methacrylate copolymer, allyl methacrylates crosspolymer, hydrophobically-modified ethoxylated urethane, and mixtures thereof.

Examples of materials which are useful for the free volume hydrophobic matrix core of the spheres of the present invention include Methacrylate Copolymer (Allianz OPT available from ISP or Aculyn PE Polymer from Rohm and Haas Company, Inc.). More specifically the preferred polymers for the free volume hydrophobic matrix core of the spheres of the present invention are acrylates/C₁₂₋₂₂ alkylmethacrylate copolymers.

The film forming hydrophilic surface of the spheres comprising water or alcohol soluble natural or synthetic polymers and copolymers, and mixtures thereof. Suitable polymers include carbohydrates, such as starch derived from different plant sources, including high amylose and high amylopectin varieties. The term “starch,” as referred to herein, is also meant to include water soluble film forming polymeric materials derived from starch including starch derivatives such as starch hydrolyzate products, modified starches, modified starch derivatives and maltodextrins. Other polymers for use in the present invention are polyvinyl alcohol, cellulose and its derivatives, polysaccharide gums and their derivatives, polyethylene glycol, water soluble acrylics, water soluble polyesters, hydroxyalkyl starches, polyvinyl pyrrolidone, polyvinyl pyrrolidone cellulose derivatives, casein, gelatin, solubilized proteins, polyacrylamide, polyamines, polyquaternary amines, styrene maleic anhydride (SMA) resins, polyethylene amine and any other conventional water soluble polymer or a combination thereof of the above-described materials.

Examples of synthetic hydrophilic polymers which are useful for the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, and water soluble polyamide or polyester.

Examples of other hydrophilic film forming materials include hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister, Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the Du Pont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides of the non-sweet, colloidally-soluble types, such as natural gums, for example, gum arabic, starch derivatives, dextrinized and hydrolyzed starches, and the like. A suitable polysaccharide is a water dispersible, modified starch commercially available as Capule®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment the natural gum is a gum arabic, commercially available from TIC Gums Inc. Belcamp, Midland.

Combinations of different polymers or similar polymers with definite molecular weight characteristics can be used in order to achieve preferred film forming capabilities, mechanical properties, and kinetics of dissolution. Considerations in the selection of the matrix material include good barrier properties to the odorous or non-odorous substances, low toxicity and irritancy, and stability.

### Odorous and Non-Odorous Malodor Counteracting Substances

Preferably, an odorous or non-odorous malodor counteracting substance is included in the controlled delivery system of the present invention. The odorous or non-odorous malodor counteracting substance that can be encapsulated in the carrier system of the present invention can be any odoriferous or non-odoriferous malodor counteracting substance and can be selected according to the desires of the fragrance creator. In general terms, such these materials are characterized by a vapor pressure below atmospheric pressure at ambient temperatures. The high boiling odorous or non-odorous malodor counteracting substance employed herein will most often be solids at ambient temperatures, but also can include high boiling liquids. A wide variety of chemicals are known for perfumery uses, including materials such as aldehydes, ketones, esters, and the like. More commonly, naturally occurring plant and animal oils and exudates comprising complex mixtures of various chemical components are known for use as fragrances, and such materials can be used herein. Fragrances useful for the present invention can be a single aroma chemical, relatively simple in their composition, or can comprise highly sophisticated, complex mixtures of natural and synthetic chemical components, all chosen to provide any desired odor.

Suitable odorous or non-odorous malodor counteracting substance which can be used in the present invention comprise, for example the high boiling components of woody/earthy bases containing exotic materials such as sandalwood oil, civet, patchouli oil, and the like. The perfumes herein can be of a light, floral fragrance, such as for example, high boiling components of rose extract, violet extract, and the like. The perfumes herein can be formulated to provide desirable fruity odors, such as for example lime, lemon, orange, and the like. The perfume can be any material of appropriate chemical and physical properties which exudes a pleasant or otherwise desirable odor when applied to fabrics. Perfume materials suitable for use in the present invention are described more fully in S. Arctander, Perfume Flavors and Chemicals, Vols. I and II, Aurthor, Montclair, N.J. and the Merck Index, 8th Edition, Merck & Co., Inc. Rahway, N.J., both references being incorporated herein by reference.

In the preferred embodiment the controlled release system of the present invention comprises from about 1% to about 50% by weight free volume hydrophobic matrix core, from about 1% to about 50% by weight hydrophilic film forming surface, from about 1% to about 80% by weight fragrance or non-odorous malodor counteracting substances, and from about 0% to about 90% by weight water. The spheres have an average particle size in the range from about 0.001 microns to about 5 microns. The spheres can be incorporated into any personal and household products, preferably in deodorants, hair products, fabric fresher, and air fresher compositions.

Polyvinyl alcohol is an excellent barrier material to the permeation of the volatile fragrance ingredients, and as a result the controlled release systems of the present invention will release the encapsulated substances at a very slow rate, depending on the composition of the matrix, such as the ratio of polyvinyl alcohol to other matrix materials. For example, the odorous or non-odorous malodor counteracting substances may diffuse at any of the rates of the following:

(i) at steady-state or zero-order release rate in which there is a substantially continuous release per unit of time;

(ii) a first-order release rate in which the rate of release declines towards zero with time; and

(iii) a delayed release in which the initial rate is slow, but then increases with time.

It has been found that a greater amount of polyvinyl alcohol in the system provides slower release rate as compared to a matrix including a lesser amount of polyvinyl alcohol in combination with a polysaccharide. For example, a matrix having about 30% to about 40% polyvinyl alcohol has a slower release rate than a matrix having about 5% to about 10% polyvinyl alcohol. For example, if a high amount of polyvinyl alcohol is used in the system, such as in the range of about 20% to about 40%, the matrix provides controlled release of the active agent over an extended period of time up to few weeks.

The controlled delivery system can be incorporated into personal and household care products, such as deodorants, body wash, hair products, shampoos, rinses, conditioners, air refresher, fabric refresher, and other personal and household products, for the counteraction and management of malodor.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### Preparation of the Multi Component Fragrance Delivery System

### Example 1

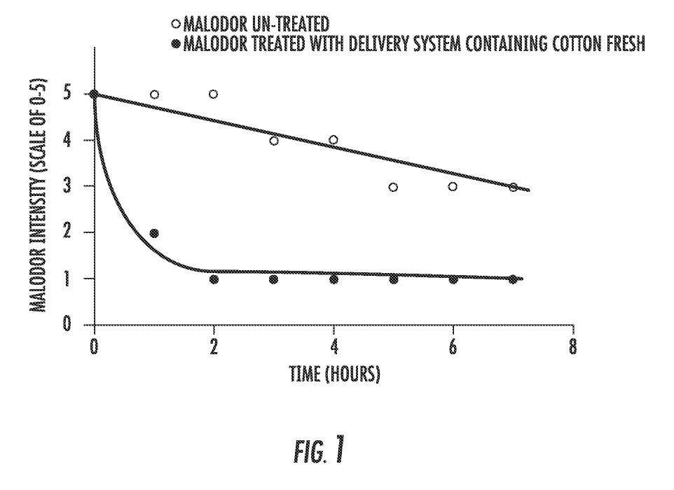

Product effectiveness tests were performed on the controlled delivery system a malodor counteracting fragrance, cotton fresh fragrance. The malodor tested was sweat and the subjects of the malodor evaluation were 8 used dirty socks that had a very strong sweaty foot odor. The malodor intensity of the socks was evaluated and malodor intensity was found to be the same for all 8 socks. Two sets of experiments were performed each with duplicates; 2 socks were left un-treated and two socks were treated with a spray comprising 1% encapsulated fragrance in the controlled release system of the present invention diluted with water. Olfactory evaluation of the malodor intensity of the socks were performed at different time intervals by 12 evaluators using an intensity scale of 0 to 5, where 5 is a very strong malodor and 0 is no residue of malodor. The data (n=12) was recorded and the results are presented in FIG. 1.

The results clearly show that 2 hours after treating the malodor with the controlled delivery system with the cotton fresh fragrance, a very faint, almost un-notable malodor was observed. Whereas the untreated malodor subject, had very strong malodor.

The socks were tested again after 24 hours and the results show that no malodor was detected on the socks treated with the controlled delivery system of the present invention. Whereas the socks treated with the free fragrance exhibited a noticeable malodor.

### Example 2

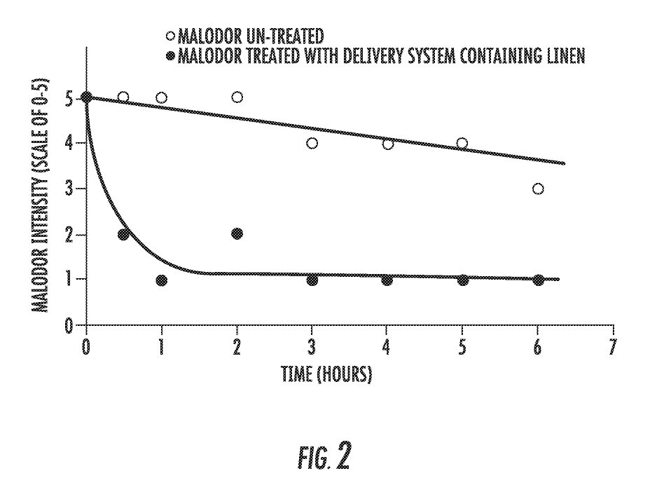

Product effectiveness tests were performed on the controlled delivery system a malodor counteracting fragrance, linen fragrance. The malodor tested was sweat and the subjects of the malodor evaluation were eight used dirty socks that had a very strong sweaty foot odor. The malodor intensity of the socks was evaluated and malodor intensity was found to be the same for all eight socks. Two sets of experiments were performed each with duplicates; two socks were left un-treated and two socks were treated with a spray comprising 1% encapsulated fragrance diluted with water. Olfactory evaluation of the malodor intensity of the socks were performed at different time intervals by 12 evaluators using an intensity scale of 0 to 5, where 5 is a very strong malodor and 0 is no residue of malodor. The data (n=12) was recorded and the results are presented in FIG. 2.

The socks were tested again after 24 hours and the results show that no malodor was detected on the socks treated with the controlled delivery system of the present invention. Whereas the socks treated with the free fragrance exhibited a noticeable malodor.

The ability of the controlled delivery system to control and eliminate malodor was also determined by analytical technique using an onion malodor, as a model malodor system. The onion malodor has many similar characteristics (same ingredients) as urine malodor, sweat, and others.

### Ability of the Controlled Delivery System to Absorb Malodor

### Example 3

The ability of the controlled delivery system of the present invention to absorb malodor was tested using headspace measurements. Headspace measurements were done utilizing Solid phase micro extraction (SPME) fiber followed by Gas Chromatography-Mass Spectra (GC-MS) analysis. Solid phase micro extraction (SPME) is an innovative, solvent free technology that is fast, economical, and versatile. SPME is a fiber coated with a liquid (polymer), a solid (sorbent), or a combination of both. The fiber coating removes the compounds from your sample by absorption in the case of liquid coatings or adsorption in the case of solid coatings. The SPME fiber is then inserted directly into the Gas Chromatograph for desorption and analysis.

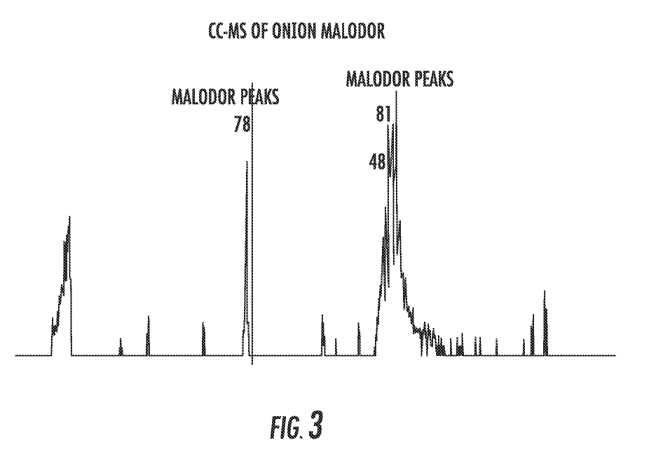

A jar containing a weighted amount of the onion malodor was equilibrated for 10 minutes. The headspace in the jar was then collected for 10 minutes and injected into the GC-MS (FIG. 3).

The retention time of the typical malodor peaks can be clearly observed at about 7.8 minutes and at 10 minutes. The amount of malodor in the headspace was determined by GC-MS.

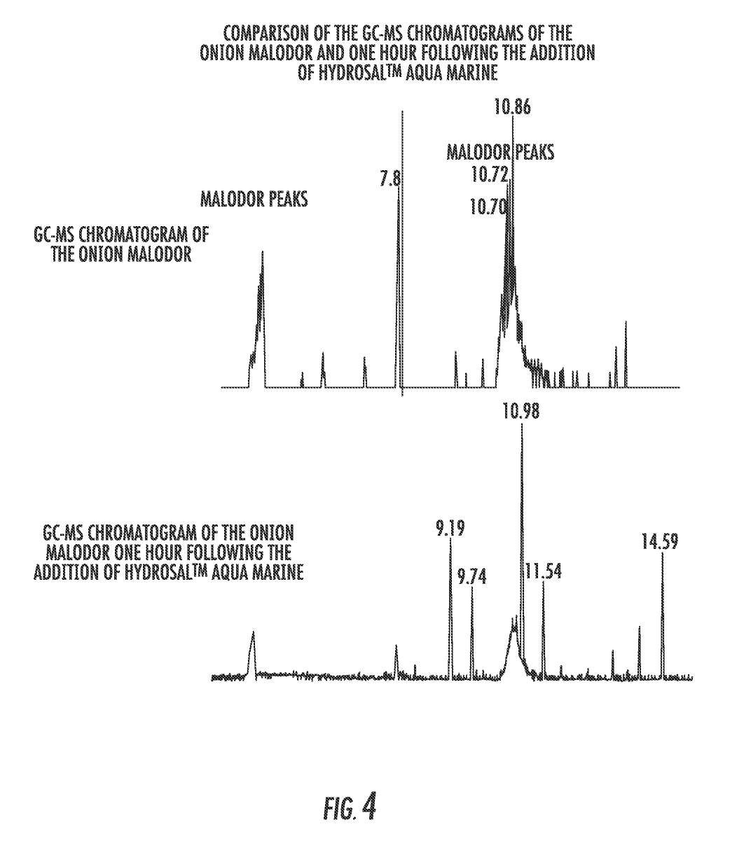

The jar containing the onion malodor was then sprayed with a known amount of the controlled delivery system with aqua marine fragrance and the sample was equilibrated for an hour. The headspace in the jar was then collected for 10 minutes and injected into the GC-MS (FIG. 4) to determine the amount of the malodor in the headspace.

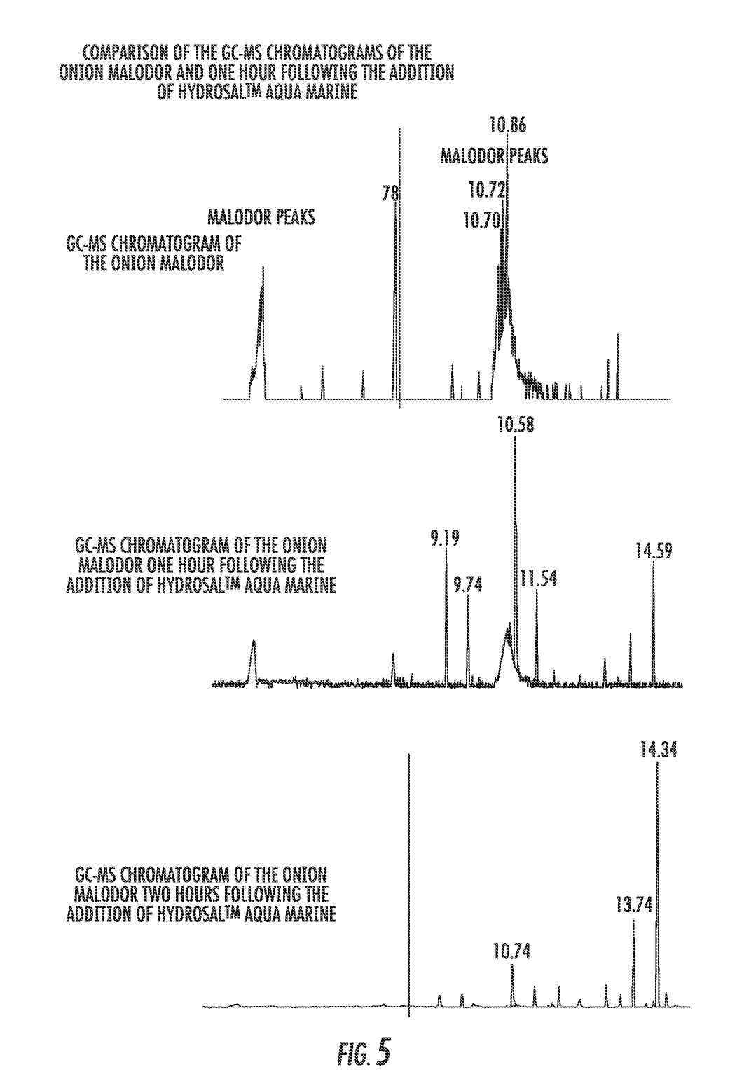

The height and the area of the typical malodor peaks, at about 7.8 minutes and at 10 minutes are significantly reduced (FIG. 4) as compared to the free malodor. The sample comprising the onion malodor sprayed with the controlled delivery system was equilibrated for 2 hour. The headspace in the jar was then collected for 10 minutes and injected into the GC-MS (FIG. 5) to determine the amount of the malodor in the headspace.

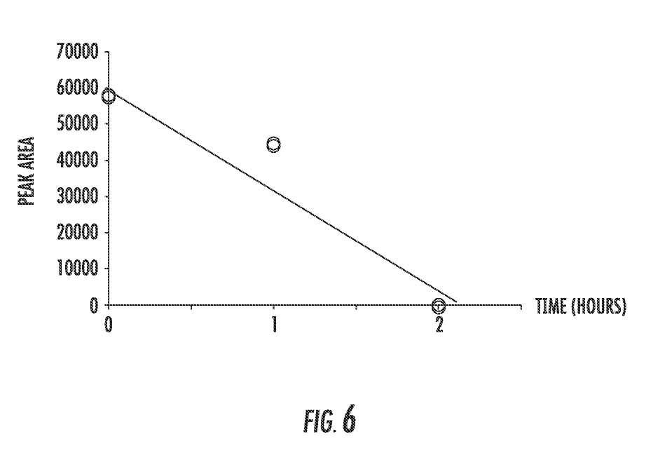

After 2 hours the malodor peaks have almost disappeared and very low malodor was detected (FIG. 6).

The results clearly demonstrate the ability of the controlled delivery system to absorb various types of malodor. The delivery system was found to absorb and neutralize malodor, an effect that was sustained over an extended period of time. The simultaneous action eliminates malodor and provided a pleasant odor that lasted for hours and in some cases days and weeks.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A dual action controlled release delivery system for management of malodor, said system comprising:
a plurality of spheres, said spheres comprising a free volume hydrophobic matrix core and a hydrophilic film forming surface surrounding said core; and
a malodor counteracting substance encapsulated in said free volume hydrophobic matrix core of said spheres,
wherein said dual action controlled release system is adapted to release said encapsulated malodor counteracting substance and thereafter absorb malodor into said matrix core.

2. The system of claim 1 wherein said malodor counteracting substance is one or more malodor counteracting fragrances.

3. The system of claim 1 wherein said malodor counteracting substance is one or more non-odorous malodor counteracting substances.

4. The system of claim 1 wherein said spheres have a size in the range of from about 0.05 microns to about 2 microns.

5. The system of claim 1 wherein said system comprises about 1% to about 50% by weight said free volume hydrophobic matrix core, from about 1% to about 50% by weight said hydrophilic film forming surface, from about 1% to about 80% by weight of said malodor counteracting substances and from about 0% to about 90% by weight water.

6. The system of claim 1 wherein said matrix core comprises about 20% to about 40% polyvinyl alcohol.

7. The system of claim 1 wherein said hydrophilic film forming surface is formed of a material selected from the group consisting of starch, polyvinyl alcohol, cellulose and its derivatives, polysaccharide gums and their derivatives, polyethylene glycol, water soluble acrylics, water soluble polyesters, hydroxyalkyl starches, polyvinyl pyrrolidone, polyvinyl pyrrolidone cellulose derivatives, casein, gelatin, solubilized proteins, polyacrylamide, polyamines, polyquaternary amines, styrene maleic anhydride (SMA) resins, and polyethylene amine and combinations thereof.

8. The system of claim 1 wherein said hydrophilic film forming surface is formed of a material selected from the group consisting of water soluble celluloses, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, and water soluble polyamide and combinations thereof.

9. The system of claim 1 wherein said hydrophilic matrix core is formed of a material selected from methacrylate copolymer, allyl methacrylates crosspolymer, hydrophobically-modified ethoxylated urethane, and mixtures thereof.

10. A personal care product for skin and hair comprising the system of claim 1.

11. The personal care product of claim 10 wherein the product is selected from deodorants, shampoos, rinses, conditioners, and body washes.

12. A household product comprising the system of claim 1.

13. The household product of claim 12 wherein the product is an air refresher.

14. A fabric care product comprising the system of claim 1.

15. The fabric care product of claim 14 wherein said product is a fabric refresher.
